(19) United States
(12) Patent Application Publication
(10) Pub. No.: US 2013/0245441 A1
(43) Pub. Date: Sep. 19, 2013

(54) Pressure-Volume with Medical Diagnostic Ultrasound Imaging

(71) Applicant: Saurabh Datta, Cupertino, CA (US)

(72) Inventor: Saurabh Datta, Cupertino, CA (US)

(73) Assignee: Siemens Medical Solutions USA, Inc., Malvern, PA (US)

(21) Appl. No.: 13/419,174

(22) Filed: Mar. 13, 2012

(51) Int. Cl. A61B 8/04

(52) U.S. Cl. 600/438

(57) ABSTRACT

Pressure-volume analysis is provided in medical diagnostic ultrasound imaging. The heart of a patient is scanned multiple times during a given cycle. B-mode and flow information are obtained for various times. The flow information is used to estimate pressure over time. A reference pressure, such as from a cuff, may be used to calibrate the pressure waveform. The B-mode information is used to determine a heart volume over time, such as a left ventricle volume over time. The heart volume over time and pressure over time are plotted, providing a pressure-volume loop. The pressure-volume loop is determined non-invasively with ultrasound.

### BACKGROUND

This present embodiments relate to medical diagnostic ultrasound. In particular, pressure-volume information is determined using ultrasound imaging.

A pressure-volume loop is used to evaluate the cardiac function of a patient. The pressure-volume loop is a load independent measure and correlates well with fundamental physiology. However, catheters are used for calculating the pressure-volume loop. Such invasive approaches are perceived to be more accurate and are used for critically ill patients.

There is continued research to define and measure image-based surrogate parameters, such as deformation, velocities, and strains that define the mechanics of the heart. For example, the left ventricle pressure or pressure waveform is measured from a radial artery or a peripheral artery over time. Given the typically limited spatial extent for real-time ultrasound scanning, the artery is used. The diastolic and systolic pressure is used to derive the pressure at the aorta. This may used as a surrogate for invasive measurement to evaluate certain clinical cardiac conditions. However, the information contained in the pressure-volume loop may potentially provide more valuable information.

### BRIEF SUMMARY

By way of introduction, the preferred embodiments described below include a method, system, computer readable medium, and instructions for pressure-volume analysis with medical diagnostic ultrasound imaging. The heart of a patient is scanned multiple times during a given cycle. Both B-mode and flow information are obtained for various times. The flow information is used to estimate pressure in the heart over time. A reference pressure, such as from a cuff, may be used to calibrate the pressure waveform. Pressure may alternatively be measured invasively. The B-mode information is used to determine a heart volume over time, such as a left ventricle volume over time. The heart volume over time and pressure over time are plotted, providing a pressure-volume loop. The pressure-volume loop is determined non-invasively with ultrasound.

In a first aspect, a method is provided for pressure-volume analysis in medical diagnostic ultrasound. B-mode and flow ultrasound data representing a three-dimensional region of a patient are acquired at a substantially same time. The acquiring is repeated multiple times in a cardiac cycle. A processor estimates pressure as a function of time at one or move valves of the heart from the flow ultrasound data. The processor calculates a volume of the three-dimensional region as a function of time from the B-mode data. A pressure-volume loop is displayed with the pressure as a function of time and the volume as a function of time. The pressure and the volume are obtained non-invasively.

In a second aspect, a non-transitory computer readable storage medium has stored therein data representing instructions executable by a programmed processor for pressure-volume analysis in medical diagnostic ultrasound. The storage medium includes instructions for receiving ultrasound data representing a patient volume at different times in a first cardiac cycle, determining pressure as a function of time from the ultrasound data, identifying a value for a heart volume as a function of time from the ultrasound data, and outputting information as a function of the pressure as a function of time and the heart volume as a function of time.

In a third aspect, a non-transitory computer readable storage medium has stored therein data representing instructions executable by a programmed processor for pressure-volume analysis in medical diagnostic ultrasound. The storage medium includes instructions for computing a cavity volume from first ultrasound data, computing differential flow from second ultrasound data, computing a pressure from the differential flow and a reference pressure, and generating a pressure to volume relationship from the pressure and the cavity volume.

In a fourth aspect, a non-transitory computer readable storage medium has stored therein data representing instructions executable by a programmed processor for pressure-volume analysis in medical diagnostic ultrasound. The storage medium includes instructions for measuring a pressure waveform representing cavity pressure, computing cavity volume as a function of time from ultrasound data. A pressure volume loop is computed by combining the pressure and volume information.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart of one embodiment of a method for pressure-volume analysis in medical diagnostic ultrasound;

FIG. 2 shows example graph of a pressure-volume loop; and

FIG. 3 is a block diagram of one embodiment of a system for pressure-volume analysis in medical diagnostic ultrasound.

### DETAILED DESCRIPTION OF THE DRAWINGS AND PRESENTLY PREFERRED EMBODIMENTS

The pressure-volume loop is estimated non-invasively for evaluation of heart patients. The pressure-volume loop may be estimated in a routine outpatient setting using volume echo imaging, allowing pressure-volume loop analysis for screening or post-procedure monitoring of patients. The pressure-volume loop may be generated automatically, avoiding variance due to operators configuring differently. Real-time, non-invasive, minimally invasive, invasive and/or automated pressure-volume loop calculation may be used in an interventional cardiac procedure, like cardiac resynchronization therapy (CRT).

Real-time volumetric B-mode, color Doppler and/or spectral Doppler data is used for identification and measurement of anatomical volumes (e.g. left ventricle (LV)) as a function of time along with flow estimated pressure differences across different valves or anatomies. The flow estimated pressure may be combined with a reference pressure measurement, such as brachial cuff pressure or an estimated aortic pressure waveform, to generate a partial or complete pressure-volume loop. The pressure and volume relationship is displayed as one or more plots for evaluation of cardiac function. Clinically or physiologically relevant parameters, such as cardiac contractility, elastance, cardiac reserve and stroke work, may be calculated from the pressure and volume information.

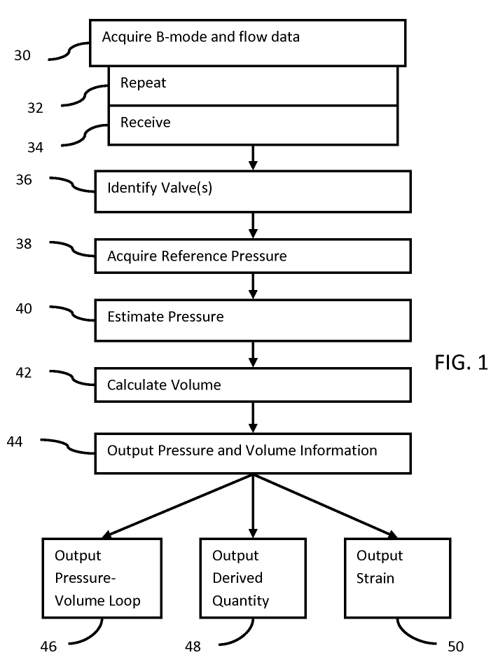

FIG. 1 shows a method for pressure-volume analysis in medical diagnostic ultrasound. The method is performed by the system 10 of FIG. 3 or a different system. The acts of FIG. 1 are performed in the order shown or a different order. Additional, different or fewer acts than shown in FIG. 1 may be used. For example, act 38 is not performed, and the ultrasound-based pressure is used without calibration to a reference. As another example, none, one, two, or different outputs than acts 46, 48, and 50 are performed. The acts of FIG. 1, described below, may be implemented in different ways. At least one example embodiment is provided below, but other embodiments are possible.

The method obtains pressure and volume information non-invasively. The pressure-volume loop may be provided without surgery. The ultrasound probe is positioned on the outside of the patient or in the patient's esophagus without surgical incision or puncturing the skin. Non-invasive acquisition allows more frequent analysis and/or analysis for patients that should not undergo a surgical procedure. In alternative embodiments, the reference pressure or ultrasound data is obtained using an invasive catheter or other intra-operative probe.

The method obtains the pressure and volume information automatically. The user may activate the method. For example, the user configures the ultrasound system to scan the patient and arranges for measurement of a reference pressure. After positioning the transducer probe to scan the heart or other location from a desired direction, the user activates acquisition of the pressure and volume information. After activation, the pressure and volume information are acquired automatically. The user does not indicate locations of the heart (e.g., ventricle or valves) in images, does not input measurements, or perform actions other than to maintain the transducer probe at the desired location for scanning the patient. In other embodiments, the method is semi-automatic. The user indicates valve, heart wall or other positions, inputs reference pressure, approves quality of information being obtained, or assists the otherwise automatic acquisition of the pressure and volume information.

The pressure and volume information are automatically acquired for the left ventricle. Alternatively, pressure and volume information are acquired for the right ventricle, both ventricles, or the whole heart. Pressure and volume may be determined for other parts of the patient.

In act 30, B-mode and flow ultrasound data are acquired. B-mode data represent intensities. Flow data represent estimates of velocity, energy (e.g., power), and/or variance. In one embodiment, at least velocity and energy are estimated. The data are acquired by scanning or from memory. The data are received in act 34 by scanning or by transfer. In one embodiment, the data are acquired during real-time scanning or as the scanning occurs.

The ultrasound data represents a volume of a patient. The volume is scanned along different planes or other distribution of scan lines within the volume. The scanned volume is an interior of an object, such as the patient. Scanning the volume provides data representing the volume, such as representing a plurality of different planes in the object (e.g., patient or heart). The data representing the volume is formed from spatial sampling of the object. The spatial samples are for locations distributed in an acoustic sampling grid in the volume. Where the acoustic sampling grid includes planar arrangements of samples, the spatial samples of the object include samples of multiple, non-planar planes or slices.

Spatial samples along one or more scan lines are received in act 34. Where the transmit beam insonifies just one receive scan line, then samples along that scan line are received. Where the transmit beam insonifies multiples scan lines, then samples along the multiple scan lines are received. For example, receive beamforming is performed along at least thirty distinct receive lines in response to one broad transmit beam. To generate the samples for different receive beams, parallel receive beamformation is performed so that the different receive beams are sampled at a same time. For example, a system may be capable of forming tens or hundreds of receive beams in parallel. Alternatively, signals received from the elements are stored and sequentially processed.

Spatial samples are acquired for a plurality of receive lines in response to one and/or in response to sequential transmit beams. Using broad beam transmission, spatial samples for multiple thin slices may be simultaneously formed using dynamic receive focusing (e.g., delay and/or phase adjust and sum). Alternatively, Fourier or other processing may be used to form the spatial samples.

The scanning may be performed a plurality of times. The acts are repeated to scan sequentially different portions of the field of view. Alternatively, performing the scanning once acquires the data for the entire field of view.

The complete volume is scanned once for B-mode, but at different times for flow. Scanning at different times acquires spatial samples associated with flow. Any now known or later developed pulse sequences may be used. A sequence of at least two (flow sample count) transmissions is provided along each scan line. Any pulse repetition frequency, ensemble/flow sample count, and pulse repetition interval may be used. The echo responses to the transmissions of the sequence are used to estimate velocity, energy (power), and/or variance at a given time. The transmissions along one line(s) may be interleaved with transmissions along another line(s). With or without interleaving, the spatial samples for a given time are acquired using transmissions from different times. The estimates from different scan lines may be acquired sequentially, but rapidly enough to represent a same time from a user perspective.

The received spatial flow samples may be wall filtered/clutter filtered. The clutter filtering is of signals in the pulse sequence for estimating motion at a given time. A given signal may be used for estimates representing different times, such as associated with a moving window for clutter filtering and estimation. Different filter outputs are used to estimate motion for a location at different times.

Flow data is generated from the spatial samples. Doppler processing, such as autocorrelation, may be used. In other embodiments, temporal correlation may be used. Another process may be used to estimate the flow data. Color Doppler parameter values (e.g., velocity, energy, or variance values) are estimated from the spatial samples acquired at different times. “Color” is used to distinguish spatial distribution of flow from spectral Doppler imaging, where the power spectrum for one or more particular range gates is estimated. The change in frequency between two samples for the same location at different times indicates the velocity. A sequence of more than two samples may be used to estimate the color Doppler parameter values. Estimates are formed for different groupings of received signals, such as completely separate or independent groupings or overlapping groupings. The estimates for each grouping represent the spatial location at a given time. Multiple frames of flow data may be acquired to represent the volume at different times.

The estimation is performed for spatial locations in the volume. For example, velocities for the different planes are estimated from echoes responsive to the scanning. In alternative embodiments, spectral Doppler data is acquired for specific locations, such as flow regions extending across a valve. In yet other embodiments, both color and spectral Doppler information are acquired, such as to use the color Doppler data to locate the valve related flow and spectral Doppler to acquire the velocities used in pressure estimation.

The flow estimates may be thresholded. Thresholds are applied to the velocities. For example, a low velocity threshold is applied. Velocities below the threshold are removed or set to another value, such as zero. As another example, where the energy is below a threshold, the velocity value for the same spatial location is removed or set to another value, such as zero. Alternatively, the estimated velocities are used without thresholding.

B-mode data is also acquired. One of the scans used for flow data estimation or a different scan is performed. The intensity of the echoes is detected for the different spatial locations.

For the volume, some spatial locations are represented by B-mode data and other locations are represented by flow data. Thresholding or another process is performed to avoid a location being represented by both B-mode and flow data. Alternatively, one or more locations may have values for both B-mode and flow data. While both types of data together represent the volume, the different types of data may be separately stored and/or processed or may be merged into one set representing the volume.

By using broad beam transmit and receiving along a plurality of scan lines or otherwise acquiring the data for a larger sub-volume or entire volume for each transmission, more rapid scanning is provided. The more rapid, repeated scanning in act 32 may allow for real-time acquisition of B-mode and color Doppler estimates. For example, the entire volume is scanned at least 10 times a second. In one embodiment, the volume rate is 20, 25 or other numbers of volumes per second. Each volume scan is associated with acquiring both B-mode and flow data. The different types of data are acquired at a substantially same time which allows for interleaving of different transmissions and/or receive processing for the different types of data. For example, ten or more volumes of data are acquired each heart cycle where each volume includes B-mode and velocity data representing a generally same portion (e.g., within 1/10^{th} of the heart cycle of each other) of the heart cycle. In alternative embodiments, the rate of acquisition for B-mode data is greater than or less than for color Doppler data and the same or less than spectral Doppler data.

By acquiring B-mode and flow data at different locations (e.g., voxels) distributed in three dimensions, real-time volumetric flow and B-mode data is acquired. Beat-to-beat full volume B-mode and/or flow acquisition capability may allow simultaneous volume and flow measurements across inflow and outflow of the heart or left ventricle. By using parallel receiving, the volumetric data may be acquired without stitching. Different transmit focal depths used sequentially to scan the entire volume may be avoided. Alternatively, stitched acquisition is used.

The volumetric data may or may not include spectral Doppler information. For example, the flow information for one, two, or more locations (e.g., valves) is spectral Doppler data representing inflow and outflow. In alternative embodiments, spatial velocity (e.g., color Doppler) is used without spectral Doppler for the valve flow.

The repetition in act 32 is through part of a heart cycle or more. For example, the repetition occurs multiple times in a same heart cycle. A sequence of volumes is acquired. Data representing the heart through one or more entire heart cycles may be obtained. Using more than one heart cycle may allow averaging. The data from different heart cycles representing a same phase may be combined or any quantities calculated from data of the same phase but different cycles may be averaged.

In one embodiment, the acquisition of act 30 of data and corresponding reception in act 34 by the system with repetition in act 32 results in B-mode data representing the left ventricle throughout at least one heart cycle. Flow data representing the left ventricle and/or just valve locations throughout the at least one heart cycle is also obtained.

In act 36, one or more valves are identified. Mitral, aortic, tricuspid, and/or pulmonary valves are identified. The valves are identified as tissue structures or flow regions adjacent or through the tissue structures. To locate the desired valves, a volume region of interest is identified from the data. The region of interest is a tissue or flow region of interest. For example, the B-mode data is used to identify a tissue structure, such as a valve or heart wall. The region of interest is positioned over, adjacent to, or at a location relative to the tissue structure. A flow region of interest spaced from the valve to cover a jet region is identified based on the location of the valve. A flow region may include a jet, flow tracts, flow surfaces, or vessel lumen. Since the flow and B-mode data are acquired as substantially the same time, the data is spatially registered and one type of data may be used to determine a region associated with another type of data. Alternatively, the volume region of interest is identified from the flow data without B-mode information, such as identifying a jet region, jet orientation or turbulent flow. In yet other embodiments, tissue motion (e.g., tissue Doppler) is used to identify the valves.

The identification is manual, semi-automated, or automated. The user may position, size and orient the region of interest. A processor may apply any algorithm to determine the region of interest, such as a knowledge-based, model, template matching, gradient-based edge detection, gradient-based flow detection, or other now known or later developed tissue and/or flow detection. For semi-automated identification, the user may indicate a tissue structure location, edge point, or other information used by a processor to determine the location, orientation, and size of the region of interest.

More than one volume region of interest may be identified. The regions of interest are identified in the same volume. For example, two flow regions of interest are identified. The flow region may be such that flow is accurate in one region and it is used to de-alias flow in the other region. The flow regions of interest are associated with conservation of mass, such as being part of a same vessel, chamber, or other flow structure. In one embodiment, a region of interest associated with a jet for an inflow tract is identified, and a region of interest associated with an outflow tract is identified. For example, the regions of interest identify the Left Ventricle Outflow tract (LVOT) and Mitral valve annulus. Flow regions associated with other structures may be identified.

The regions of interest are spatially distinct. For overlapping or for entirely spatially distinct regions of interest, some locations in one region of interest are not in another region of interest and some locations of the other region of interest are not in the one region of interest.

In other embodiments, the different regions of interest are associated with a same tissue or flow structure. For example, two flow regions on opposite sides of a tissue structure, such as a valve, are identified. The regions of interest may be in the same flow tract to provide multiple measurements of the same flow at different locations. The regions may serve as locations for additional measurement, such as PW or spectral Doppler measurement, and their known spatial location and orientation with respect to the flow anatomy may be used to correct flow estimation.

Given the repetition, the regions of interest (e.g., valves) are tracked through the sequence. A similarity calculation may be used to determine a best fit location and orientation for a region of interest in other volumes. The correlation, minimum sum of absolute differences or other similarity calculation is performed. The B-mode data is used to track.

Alternatively, flow data is used. Both B-mode and flow data may be used, such as tracking with both and averaging the location. Rather than tracking, the identification of the valves may be performed for each volume or phase of the heart cycle independently of the identification for other phases or volumes.

In act 38, a reference pressure is acquired. The reference pressure is an actual blood pressure. For example, a brachial cuff is used to determine one or two pressures. For example, pressure in the artery at both diastole and systole are measured. Radial tonometry may be used. In other embodiments, pressure within the heart or left ventricle is directly measured using an invasive catheter.

The reference pressure is for one or more parts of the heart cycle. Direct measurement may allow pressure to be measured over time or for many phases of the heart cycle. Cuff or tonometry may provide pressure for only one or two phases.

In act 40, the pressure throughout the heart cycle or portion of a heart cycle is estimated. The pressure may be estimated using invasive or minimally invasive approaches. For example, a catheter or other device is inserted into the patient to measure pressure. Using ECG, triggering, or timestamps, the pressure measurement is temporally synchronized at acquisition or after acquisition with the ultrasound data used for volume determination. Where direct pressure measurement is not available, the pressure over time is estimated from ultrasound data. A processor calculates the pressure from velocity or other flow information.

The pressure may be an actual pressure, such as computed from differential flow calibrated by the reference pressure. Alternatively, the pressure may be a relative pressure. Using just pressure estimated from the ultrasound data, such as differential flow, relative pressure throughout the cycle is estimated. This estimated pressure provides change in pressure over time, but not the actual pressure over time.

The pressure is computed as a differential pressure. The difference in flow between the inflow and outflow tracks indicates the pressure. By identifying velocities at different valves, the difference in velocity indicates pressure. Spatial flow (e.g., color Doppler) is used. The peak velocity over a region, the center velocity of the flow region at the valve, an average velocity in the valve region, or other velocity is used.

In another embodiment, spectral Doppler velocities are used. Range gates are positioned to cover the diameter of the flow through the valves, region of maximum flow, center of flow through the valve or other location related to the valve. The range gates extend on both sides of the valve or may be positioned on just one side. The peak, average or other velocity from the spectrum is used for determining differential flow. With sufficient temporal resolution, the velocities from two or more spectra may be averaged.

In alternative embodiments, a velocity related flow quantity is used instead of velocity. For example, the volume flow through the valve or variance of flow in the jet may be used.

The difference in velocity or other flow quantity is calculated. Any function for estimating pressure may be used. For example, Bernoulli or Navier-stokes equations are used. The pressure difference across multiple valves is estimated as a function of time using known fluid mechanics principles. In one embodiment, the square of the velocity difference between the inflow and outflow tracts times a constant is used as the estimate of pressure difference across the valve or cavity. In an alternative embodiment, the velocity at a single valve is used instead of the differential velocity or flow. The difference between entry and exit velocity of one valve may be used.

The pressure estimated from the differential flow provides a differential pressure. Other approaches to estimate the flow through the inflow and outflow valves may be used.

Where a reference pressure is available, the differential pressure estimated from the ultrasound flow data may be calibrated. By scaling the estimated pressure, a more accurate pressure as a function of time may be provided.

Since the reference pressure may be for less than all the phases of interest in the heart cycle, the estimate of pressure from the velocities for the other phases is used. The ultrasound data may be used to estimate pressure at many times or phases during a heart cycles, such as at ten or more times. The reference pressure for one or two of these times is used to calibrate the estimated pressures throughout the cycle. The computed pressure differential from the reference measurement of the blood pressure (e.g., central or aortic) is used to generate a pressure waveform as a function of time. For example, a difference between the pressure estimated from flow and the reference pressure representing a same point in the cycle is determined. The same difference is applied to the flow estimated pressures for other times in the cycle. Where reference pressures are available for multiple phases, the average difference is used. Alternatively, the amount of difference to be used for calibration is interpolated as a function of time and applied to the flow estimated pressures. The calibrated pressure is used to scale the pressures for other times in the heart cycle.

The pressure waveform in different cavities of heart may be estimated separately (e.g. at different times). The different estimates may then be combined to generate one pressure volume curve. Different segments of the PV loop are computed at different times. The different segments may be combined or used individually as needed

In act 42, the volume is calculated. The volume is of a three-dimensional region. The volume for any region is used. For example, the volume of the left ventricle is determined. The volume of the right ventricle, the entire heart, or other cavities may be calculated.

The volume is calculated from the B-mode data. Edges, tissue structures, or other information is extracted from the B-mode data. In alternative or additional embodiments, the volume is calculated from flow data. For example, the volume of a flow region, such as a large pool of blood, is determined.

Any volume determination may be used. In one embodiment, the processor automatically calculates the volume from the ultrasound data by segmenting the heart or heart cavity. The edges or heart walls for the left ventricle are found and lines connected for any gaps. Any approach may be used for automatic, semi-automatic, or manual segmentation of a heart cavity. For automatic, a processor may apply any algorithm to segment, such as a knowledge-based, model, template matching, gradient-based edge detection, gradient-based flow detection, or other now known or later developed tissue or flow detection. For example, a threshold process is used to determine whether sufficient flow exists in combination B-mode and color Doppler images. The B-mode, velocity, energy, and/or other information are thresholded. Locations with large B-mode or small velocity and/or energy are indicated as tissue. Locations with small B-mode or sufficient velocity and/or energy are indicated as flow. After low pass filtering for fill holes, the largest continuous flow region surrounded by tissue other than the valves is identified, such as using region growing, skeletonization, filtering, or directional filtering.

In one embodiment, the B-mode data for the region of interest is low pass filtered to fill in noise related holes. Gradients of the filtered B-mode data are used to determine a tissue border. The border separates tissue from flow structure. Other edge detection may be used, such as gradient of flow data to better isolate the flow of interest. Combinations of both may be used.

In another embodiment, a knowledge-based system is used. Machine learning or other training is used to determine a matrix of weights for various feature inputs to identify the cavity. The matrix represents a probability mapping of a model of the heart or cavity to the B-mode and/or flow data. The model is scaled, rotated and translated using the probability mapping to best fit to the data for a given patient. The model is annotated to indicate the location for which volume is then calculated. The volume is determined from the model after fitting.

Once segmented, the volume of the heart cavity, such as the left ventricle, is calculated. The volume is for within the tissue boundary, of the contiguous flow region, or other designation of the left ventricle or other cavity. Using the scan parameters, the spatial distribution of the B-mode or flow data, whether in a scan format, scan converted format, or interpolated to the three-dimensional grid, is used to calculate the volume.

The volume is calculated for different times during the heart cycle. In one embodiment, segmentation and volume calculation are performed separately for each acquired volume of B-mode data. In other embodiments, the segmented region is tracked or fit to subsequent or earlier volumes. Once fit to the data of other scans, the volume for the different time of the other scan is calculated based on another fitting at a different time. By calculating the volume for different phases or times in the heart cycle, the volume is determined as a function of time. The three-dimensional beat-to-beat change in cardiac cavity volume is represented as a waveform.

In act 44, information is output based on the pressure and volume. The outputs may be separate, such as displaying the pressure as a function of time and the volume as a function of time in different graphs. Values may be output as text, such as systolic and diastolic pressures and volumes. The output may include one or more images, such as a multiplanar reconstruction or three-dimensional rendering using the B-mode or flow data. The volume, valves, pressure measurement location, or other aspects of the heart may be highlighted, such as colored or represented in a graphic overlay.

Average or instantaneous values of the pressure and volume may be output, such as indicating the pressure and volume for each image in a sequence of images. Alternatively or additionally, the output shows the pressure and/or volume as a function of time. A graph, variation statistic, or other parameter representing one or more characteristics of the pressure and/or volume waveforms may be displayed.

The pressure and volume information may be displayed together, such as in a same graph or adjacent graphs to show the relationship between pressure and volume. For example, the pressure and volume waveforms are overlaid on each other with a common time axis.

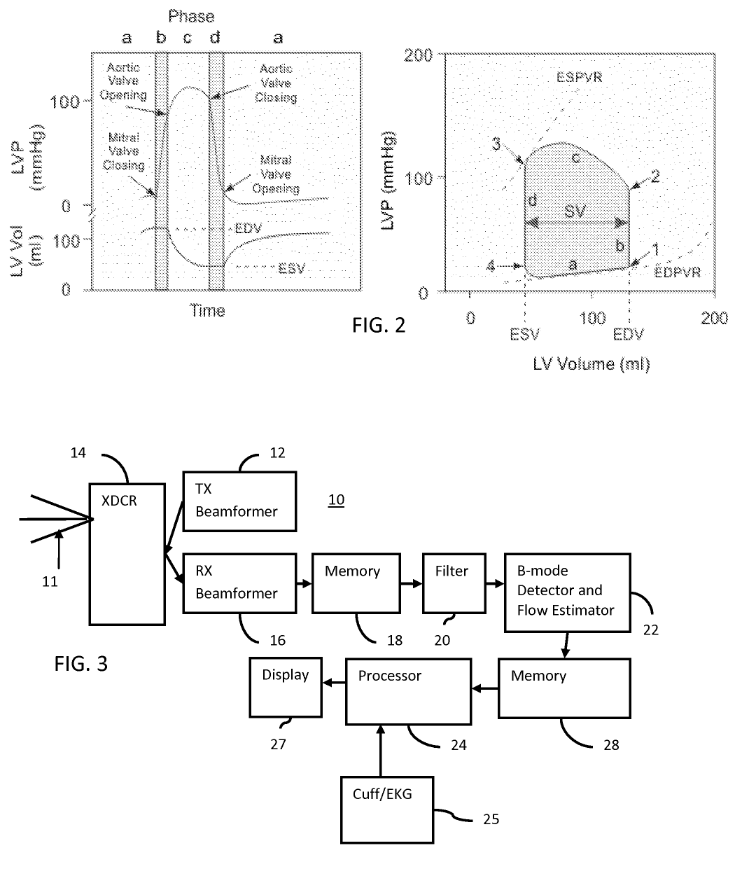

In one embodiment, a pressure-volume loop is generated in act 46. The pressure volume loop is one type of output for act 44. FIG. 2 shows an example pressure-volume loop where volume is plotted along the x-axis and pressure is plotted along the y-axis. As the volume changes, the pressure also changes. The loop represents a given heart cycle. The pressure and volumes at different times during the heart cycle are plotted on the graph. Any gaps may be interpolated or filled by fitting a curve, line, or model.

The generated graph of the pressure-volume loop is displayed. The graph is displayed during the acquiring, such as during plotting sequentially through a same heart cycle or as displaying the completed graph in a subsequent heart cycle or same imaging session. The graph represents the pressure and volume as a function of time. By combining the pressure and volume waveforms, cardiac function may be evaluated. The graph of the pressure as a function of volume synchronized by the time (e.g., EKG or acquisition synchronization) may be diagnostically useful. The pressure-volume loop is provided without invasive surgery.

In act 48, a value for a parameter is output. This value is another example of the output of act 44. The value is derived from the pressure and/or volume information, either instantaneous or as a function of time. For example, beat-to-beat parameters, such as stroke volume (SV), contractility (e.g., ejection fraction, SV/EDV, and/or dp/dt Max), preload (EDV or EDP), afterload (aortic and ventricular pressure), compliance (dV/dP), ventricular stiffness (inverse of compliance), and/or elastance (dP/dV), are calculated. As another example, parameters derived from ESPVR and EDPVR, such as PVA Pressure-volume area and/or PE Potential energy, are calculated. In yet another example, processed parameters, such as ESPVR end-systolic pressure-volume relationship, EDPVR end-diastolic pressure-volume relationship, PRSW Preload-recruitable stroke work, DPdtmax vs Ved dPdt max against end-diastolic volume relationship, and/or Emax maximal elastance (computed from the time-varying elastance data), are calculated. The stroke work (area of PVL), cardiac reserve, contractility, peak power, and/or dP/dt may be calculated from the pressure-volume loop and output. For example, LV function—CO, SV, EDV, ESV, LVEF, ESP, EDP, dP/dtmax and dP/dtmin, Stroke Work=area of PVL, LVES Elastance (EES)=ESP/ESV, LVED Stiffness (EED)=EDP/EDV, LV Effective Arterial Elastance (EA)=ESP/SV, V-A coupling=EES/EA, and/or Time Varying Wall Stress (WS(t))=P(t)*[1+3*V(t)/LVM] are output. Any clinically or physiologically relevant parameters may be calculated and displayed. Present, real-time functional information of the ventricle, the contractility state, contractility reserve, stroke work, peak power and load independent measurement of function may be obtained non-invasively in an outpatient setting.

The quantity (i.e., value) is displayed with or without images. The quantity is displayed as a value, number, graph, color modulation, or text. As a sequence of images is viewed, the quantities associated with the given volume or data are displayed.

In act 50, strain information is output with the pressure-volume loop. Strain or strain rate is another example output of act 44. Ultrasound is used to measure the strain along the scan axes or lines. Two or three-dimensional strain may be calculated. Other two or three-dimensional mechanics information may be output for comprehensive analysis of cardiac function

In a real-time implementation, the pressure and volume information is calculated during a same heart cycle as the acquisition of act 30. Before a complete heart cycle occurs after acquisition of the volume, the quantity is calculated. The calculation occurs during the cardiac cycle. Greater or lesser delay may be provided. The calculation is performed during acquisition, even if not within a same heart cycle. The calculation is part of the on-going diagnostic examination or scan session. During a subsequent heart cycle, the pressure-volume loop from a prior heart cycle is displayed. The prior heart cycle may be the immediately prior cycle or another earlier cycle. In alternative embodiments, the calculation is performed for data acquired during a different hour, day or other time, such as during a review session after an examination or scan session.

The pressure-volume loop may be used for evaluation of systolic and diastolic LV function, valve disease, heart failure, Inotropic state or other conditions. The use is during a clinical visit, as part of cardiac surgery procedures, or for evaluation and monitoring of pharmacological manipulation of heart function. The pressure-volume loop may be generated for pre-, during-, and post-operative assessment of LV function. Better quantification of dyssynchrony along with other echo based measurements may be provided for cardiac resynchronization therapy cases.

FIG. 3 shows one embodiment of a system 10 for pressure-volume analysis in medical diagnostic ultrasound. The system 10 includes a transmit beamformer 12, a transducer 14, a receive beamformer 16, a memory 18, a filter 20, a B-mode detector and flow estimator 22, a memory 28, a processor 24, a cuff/EKG input or device 25, and a display 27. Additional, different or fewer components may be provided. For example, the system includes the B-mode detector and flow estimator 22 and processor 24 without the front-end components, such as the transmit and receive beamformers 12, 16. In one embodiment, the system 10 is a medical diagnostic ultrasound system. In an alternative embodiment, the system 10 is a computer or workstation. In yet another embodiment, the B-mode detector and flow estimator 22 are part of a medical diagnostic ultrasound system or other medical imaging system, and the processor 24 is part of a separate workstation or remote system.

The transducer 14 is an array of a plurality of elements. The elements are piezoelectric or capacitive membrane elements. The array is configured as a one-dimensional array, a two-dimensional array, a 1.5D array, a 1.25D array, a 1.75D array, an annular array, a multidimensional array, a wobbler array, combinations thereof, or any other now known or later developed array. The transducer elements transduce between acoustic and electric energies. The transducer 14 connects with the transmit beamformer 12 and the receive beamformer 16 through a transmit/receive switch, but separate connections may be used in other embodiments.

The transmit and receive beamformers 12, 16 are a beamformer for scanning with the transducer 14. The transmit beamformer 12, using the transducer 14, transmits one or more beams to scan a region. Vector®, sector, linear or other scan formats may be used. In one embodiment, the transmit beamformer 12 transmits beams sufficiently large to cover at least thirty distinct receive lines, and the receive beamformer 16 receives along these distinct receive lines in response to the transmit beam. Use of the broad beam transmit and parallel receive beamforming along tens or hundreds of receive lines allows for real-time scanning of multiple slices or a volume, such as of the left ventricle. The receive lines and/or transmit beams are distributed in the volume, such as the receive lines for one transmit being in at least two different planes. The receive beamformer 16 samples the receive beams at different depths. Sampling the same location at different times obtains a sequence for flow estimation.

In one embodiment, the transmit beamformer 12 is a processor, delay, filter, waveform generator, memory, phase rotator, digital-to-analog converter, amplifier, combinations thereof, or any other now known or later developed transmit beamformer components. In one embodiment, the transmit beamformer 12 digitally generates envelope samples. Using filtering, delays, phase rotation, digital-to-analog conversion and amplification, the desired transmit waveform is generated. Other waveform generators may be used, such as switching pulsers or waveform memories.

The transmit beamformer 12 is configured as a plurality of channels for generating electrical signals of a transmit waveform for each element of a transmit aperture on the transducer 14. The waveforms are unipolar, bipolar, stepped, sinusoidal or other waveforms of a desired center frequency or frequency band with one, multiple or fractional number of cycles. The waveforms have relative delay and/or phasing and amplitude for focusing the acoustic energy. The transmit beamformer 12 includes a controller for altering an aperture (e.g. the number of active elements), an apodization profile (e.g., type or center of mass) across the plurality of channels, a delay profile across the plurality of channels, a phase profile across the plurality of channels, center frequency, frequency band, waveform shape, number of cycles and combinations thereof. A transmit beam focus is generated based on these beamforming parameters.

The receive beamformer 16 is a preamplifier, filter, phase rotator, delay, summer, base band filter, processor, buffers, memory, combinations thereof or other now known or later developed receive beamformer components. The receive beamformer 16 is configured into a plurality of channels for receiving electrical signals representing echoes or acoustic energy impinging on the transducer 14. A channel from each of the elements of the receive aperture within the transducer 14 connects to an amplifier and/or delay. An analog-to-digital converter digitizes the amplified echo signal. The digital radio frequency received data is demodulated to a base band frequency. Any receive delays, such as dynamic receive delays, and/or phase rotations are applied by the amplifier and/or delay. A digital or analog summer combines data from different channels of the receive aperture to form one or a plurality of receive beams. The summer is a single summer or cascaded summer. In one embodiment, the beamform summer is operable to sum in-phase and quadrature channel data in a complex manner such that phase information is maintained for the formed beam. Alternatively, the beamform summer sums data amplitudes or intensities without maintaining the phase information.

The receive beamformer 16 is operable to form receive beams in response to the transmit beams. For example, the receive beamformer 16 receives one, two, or more (e.g., 32, 48, or 56) receive beams in response to each transmit beam. The receive beams are collinear, parallel and offset or nonparallel with the corresponding transmit beams. The receive beamformer 16 outputs spatial samples representing different spatial locations of a scanned region. Once the channel data is beamformed or otherwise combined to represent spatial locations along the scan lines 11, the data is converted from the channel domain to the image data domain. The phase rotators, delays, and/or summers may be repeated for parallel receive beamformation. One or more of the parallel receive beamformers may share parts of channels, such as sharing initial amplification.

For imaging motion, such as tissue motion or fluid velocity, multiple transmissions and corresponding receptions are performed for a substantially same spatial location. Phase changes between the different receive events indicate the velocity of the tissue or fluid. A velocity sample group corresponds to multiple transmissions for each of a plurality of scan lines 11. The number of times a substantially same spatial location, such as a scan line 11, is scanned within a velocity sample group is the velocity sample count. The transmissions for different scan lines 11, different velocity sample groupings or different types of imaging may be interleaved. The amount of time between transmissions to a substantially same scan line 11 within the velocity sample count is the pulse repetition interval or pulse repetition frequency. Pulse repetition interval is used herein, but includes the pulse repetition frequency.

The memory 18 is video random access memory, random access memory, removable media (e.g. diskette or compact disc), hard drive, database, corner turning memory or other memory device for storing data or video information. In one embodiment, the memory 18 is a corner turning memory of a motion parameter estimation path. The memory 18 is operable to store signals responsive to multiple transmissions along a substantially same scan line. The memory 22 is operable to store ultrasound data formatted in an acoustic grid, a Cartesian grid, both a Cartesian coordinate grid and an acoustic grid, or ultrasound data representing a volume in a three-dimensional grid.

The filter 20 is a clutter (e.g., wall) filter, finite impulse response filter, infinite impulse response filter, analog filter, digital filter, combinations thereof or other now known or later developed filter. In one embodiment, the filter 20 includes a mixer to shift signals to baseband and a programmable low pass filter response for removing or minimizing information at frequencies away from the baseband. In other embodiments, the filter 20 is a low pass, high pass or band pass filter. The filter 20 identifies velocity information from slower moving tissue as opposed to fluids or alternatively reduces the influence of data from tissue while maintaining velocity information from fluids. The filter 20 has a set response or may be programmed, such as altering operation as a function of signal feedback or other adaptive process. In yet another embodiment, the memory 18 and/or the filter 20 are part of the flow estimator 22. A by-pass may be provided for B-mode detection.

The B-mode detector and flow estimator 22 is a Doppler processor or cross-correlation processor for estimating the flow data and a B-mode detector for determining the intensity. In alternative embodiments, another device now known or later developed for estimating velocity, energy, and/or variance from any or various input data may be provided. The flow estimator 22 receives a plurality of signals associated with a substantially same location at different times and estimates a Doppler shift frequency, based on a change or an average change in phase between consecutive signals from the same location. Velocity is calculated from the Doppler shift frequency. Alternatively, the Doppler shift frequency is used as a velocity. The energy and variance may also be calculated.

Flow data (e.g., velocity, energy, or variance) is estimated for spatial locations in the scan volume from the beamformed scan samples. For example, the flow data represents a plurality of different planes in the volume as spatial Doppler data.

The flow estimator 22 may apply one or more thresholds to identify sufficient motion information. For example, velocity and/or energy thresholding for identifying velocities is used. In alternative embodiments, a separate processor or filter applies thresholds. The B-mode detector and flow estimator 22 outputs B-mode and flow data for the volume.

The flow estimator 22 is alternatively or additionally a spectral Doppler processor. The multiple samples for each location are Fourier transformed. The resulting spectrum indicates the power at each frequency, providing an indication of velocity, energy, and variance.

The memory 28 is video random access memory, random access memory, removable media (e.g. diskette or compact disc), hard drive, database, or other memory device for storing B-mode and flow data. The stored data is in a polar or Cartesian coordinate format. The memory 28 is used by the processor 24 for the various filtering, rendering passes, calculations or other acts described for FIG. 1. The processor 24 may additionally reformat the data, such as interpolating the data representing the volume to a regularly spaced Cartesian coordinate three-dimensional grid.

The cuff or EKG connection or device 25 provides inputs for determining the pressure-volume loop. For example, a brachial cuff with a processor or output connection for measurement of the reference pressure is provided. The measurement from a device may be received by an ultrasound system. The measurement may be automated so that the reference pressure is measured as needed. Alternatively, the user may trigger measurement or even input a manually measured pressure.

Alternatively or additionally, the cuff or EKG connection or device 25 is an EKG system. The EKG signals may be used to indicate the heart phase associated with acquired data. By using EKG signals, data and/or derive quantities from different cycles but the same phase may be combined. The EKG signals may be used to synchronize the pressure and volume information instead of substantially simultaneous acquisition and time stamping.

The display 27 is a CRT, LCD, plasma, projector, monitor, printer, touch screen, or other now known or later developed display device. The display 27 receives RGB or other color values and outputs an image. The image may be gray scale or color image. The image represents the region of the patient scanned by the beamformer and transducer 14 and/or may include a pressure-volume loop or other derived quantity.

The processor 24 is a digital signal processor, a general processor, an application specific integrated circuit, field programmable gate array, control processor, digital circuitry, analog circuitry, graphics processing unit, combinations thereof or other now known or later developed device for implementing calculations, algorithms, programming or other functions. The processor 24 operates pursuant to instruction provided in the memory 18, 28, or a different memory for pressure-volume analysis with medical diagnostic ultrasound.

The processor 24 receives B-mode and flow data from the B-mode detector and flow estimator 22, the memory 28, and/or another source. In one embodiment, the processor 24 implements one or more of the algorithms, acts, steps, functions, methods or processes discussed herein, by processing the data and/or controlling operation of other components of the system 10. Additional or multiple processors may be used to implement various aspects of the algorithms.

The processor 24 is configured by software and/or hardware. The processor 24 causes acquisition of B-mode and flow data. Alternatively or additionally, the processor 24 controls reception of the data. The processor 24 controls measurement or reception of the reference pressure and/or EKG signal. The processor 24 processes the data to identify valves, estimate pressure, calculate volume and generate the output (e.g., pressure volume loop graph).

The instructions for implementing the processes, methods and/or techniques discussed above are provided on non-transitory computer-readable storage media or memories, such as a cache, buffer, RAM, removable media, hard drive or other computer readable storage media. In one embodiment, the instructions are for pressure-volume analysis in medical diagnostic ultrasound. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for pressure-volume analysis in medical diagnostic ultrasound, the method comprising:
acquiring B-mode and flow ultrasound data representing a three-dimensional region of a patient at a substantially same time;
repeating the acquiring multiple times in a cardiac cycle;
estimating, with a processor, pressure as a function of time at one or more valves of the heart from the flow ultrasound data;
calculating, with the processor, a volume of the three-dimensional region as a function of time from the B-mode data; and
displaying a pressure-volume loop with the pressure as a function of time and the volume as a function of time, the pressure and the volume being obtained non-invasively.

2. The method of claim 1 wherein repeating comprises repeating the acquiring with a three-dimensional region frame rate of at least 10 per second including interleaved scans for both the B-mode and flow ultrasound data.

3. The method of claim 1 wherein acquiring comprises acquiring the data representing a heart of the patient, the flow ultrasound data comprising velocity data at different voxels;
further comprising:
identifying the one or more valves from the velocity data; and
obtaining spectral Doppler data from adjacent to the one or more valves;
wherein estimating the pressure comprises estimating with the spectral Doppler data.

4. The method of claim 1 wherein estimating the pressure comprises calculating differential pressure across the one or more valves from velocity.

5. The method of claim 4 further comprising:
acquiring a reference pressure;
wherein estimating the pressure as a function of time comprises calibrating the differential pressure at a first time to the reference pressure and scaling the reference pressure at other times with the calibrating.

6. The method of claim 1 wherein calculating the volume comprises:
automatically segmenting the volume of a heart cavity; and
calculating the volume of the heart cavity based on the segmenting.

7. The method of claim 1 wherein displaying comprises generating a graph of the pressure as a function of volume synchronized by the time.

8. The method of claim 1 further comprising:
calculating a stroke work, afterload, cardiac reserve, contractility, peak power, compliance, elastance, ventricular stiffness, pressure-volume area, end diastolic- and end systolic-pressure volume relationship, dP/dt or combinations thereof.

9. The method of claim 1 wherein acquiring, repeating, estimating, calculating, and displaying are performed automatically for a left ventricle, a right ventricle, or both the left and right ventricles and without user input for location indication.

10. The method of claim 1 further comprising:
displaying strain information with the pressure-volume loop.

11. In a non-transitory computer readable storage medium having stored therein data representing instructions executable by a programmed processor for pressure-volume analysis in medical diagnostic ultrasound, the storage medium comprising instructions for:
receiving ultrasound data representing a patient volume at different times in a first cardiac cycle;
determining pressure as a function of time from the ultrasound data;
identifying a value for a heart volume as a function of time from the ultrasound data; and
outputting information as a function of the pressure as a function of time and the heart volume as a function of time.

12. The non-transitory computer readable storage medium of claim 11 wherein receiving comprises receiving B-mode data representing a left ventricle and flow data representing a valve of the left ventricle, wherein determining the pressure comprises determining from the flow data, and wherein identifying the value for the heart volume comprises identifying the value of the left ventricle from the B-mode data.

13. The non-transitory computer readable storage medium of claim 11 wherein determining the pressure comprises determining from velocity.

14. The non-transitory computer readable storage medium of claim 13 wherein determining the pressure comprises scaling the pressure from the velocity based on a reference pressure.

15. The non-transitory computer readable storage medium of claim 11 wherein identifying the value comprises the programmed processor calculating the value from the ultrasound data and without user input.

16. The non-transitory computer readable storage medium of claim 11 wherein outputting the information comprises outputting a pressure-volume loop without measurement from an invasive procedure.

17. The non-transitory computer readable storage medium of claim 11 wherein outputting information comprises outputting stroke work, afterload, cardiac reserve, contractility, peak power, compliance, elastance, ventricular stiffness, pressure-volume area, end diastolic- and end systolic-pressure volume relationship, dP/dt or combinations thereof

18. In a non-transitory computer readable storage medium having stored therein data representing instructions executable by a programmed processor for pressure-volume analysis in medical diagnostic ultrasound, the storage medium comprising instructions for:
computing a cavity volume from first ultrasound data;
computing differential flow from second ultrasound data;
computing a pressure from the differential flow and a reference pressure; and
generating a pressure to volume relationship from the pressure and the cavity volume.

19. The non-transitory computer readable storage medium of claim 18 further comprising:
acquiring the first and second ultrasound data representing a heart volume of a patient at multiple times during a cardiac cycle;
wherein computing the cavity volume comprises computing a left ventricle volume from B-mode data, and wherein computing differential flow comprises computing velocity at a valve of the left ventricle from spectral Doppler data.

20. The non-transitory computer readable storage medium of claim 18 wherein computing the pressure comprises calculating a differential pressure from the differential flow, and calibrating the differential pressure with the reference pressure, the pressure comprising the calibrated differential pressure.

21. The non-transitory computer readable storage medium of claim 18 wherein generating comprises generating a graph of a pressure-volume loop.

22. In a non-transitory computer readable storage medium having stored therein data representing instructions executable by a programmed processor for pressure-volume analysis in medical diagnostic ultrasound, the storage medium comprising instructions for:
measuring a pressure waveform representing cavity pressure;
computing cavity volume as a function of time from ultrasound data; and
generating a pressure volume loop combining the pressure and volume information.

23. The non-transitory computer readable storage medium of claim 22 wherein measuring comprises measuring invasively synchronized with acquisition of the ultrasound data used for computing the cavity volume.
